(19) United States
(12) Patent Application Publication
(10) Pub. No.: US 2019/0192552 A1
(43) Pub. Date: Jun. 27, 2019

(54) PHARMACEUTICAL COMPOSITIONS AND METHODS FOR TREATING KELOIDS, HYPERTROPHIC SCARS AND WOUNDS, AND FOR PROVIDING IMPROVED SKIN CARE

(71) Applicant: Cormedix Inc., Berkeley Heights, NJ (US)

(72) Inventors: Robert DiLuccio, Haymarket, VA (US); Z. Paul Lorenc, New York, NY (US)

(21) Appl. No.: 16/230,457

(22) Filed: Dec. 21, 2018

Related U.S. Application Data
(60) Provisional application No. 62/608,890, filed on Dec. 21, 2017.

(51) Int. Cl. A61K 31/726; A61K 31/549; A61K 9/08; A61K 9/06; A61K 9/14; A61P 17/02

(52) U.S. Cl. CPC A61K 31/726; A61K 31/549; A61K 9/08; A61K 9/0019; A61K 9/143; A61P 17/02; A61K 9/06

(57) ABSTRACT

A method for treating keloids, hypertrophic scars and/or wounds and/or other skin conditions, the method comprising: delivering a pharmaceutically effective amount of a pharmaceutical composition to the keloids, hypertrophic scars and/or wounds and/or other skin conditions, wherein the pharmaceutical composition comprises a mixture of cross-linked glycosaminoglycans and taurolidine and/or one or more taurolidine derivatives.

### REFERENCE TO PENDING PRIOR PATENT APPLICATION

This patent application claims benefit of pending prior U.S. Provisional Patent Application Ser. No. 62/608,890, filed Dec. 21, 2017 by Cormedix Inc. and Robert DiLuccio et al. for PHARMACEUTICAL COMPOSITIONS AND METHODS FOR TREATING KELOIDS, HYPERTROPHIC SCARS AND WOUNDS, which patent application is hereby incorporated herein by reference.

### FIELD OF THE INVENTION

This invention relates generally to pharmaceutical compositions and methods for treating a patient, and more particularly to pharmaceutical compositions and methods for treating keloids, hypertrophic scars and wounds, and for providing improved skin care.

### BACKGROUND OF THE INVENTION

1. Keloids and Hypertrophic Scars in General

A keloid is a firm, benign, frequently pruritic or painful nodular or protuberant scar that commonly develops after injuries in certain population groups. Keloids typically project beyond the site of the original injury and contain abnormally thickened collagen bundles.

A hypertrophic scar is a raised scar that may develop in any population group and usually occurs in areas of high tension such as the presternum, shoulders, knees and ankles.

2. Clinical Description of Keloids and Hypertrophic Scars

Keloids and hypertrophic scars are thickened and raised, and are caused by an increase in the total amount of collagen that is normally present in the tissue.

Keloids may develop at virtually any anatomic site, usually after minor injuries. They are uncommon in the hands, feet, eyelids, genitalia and mucous membranes. They usually develop on the upper trunk, arms and neck, especially after burn injuries, surgical procedures and vaccinations. Keloids can even form “spontaneously”, without prior injury, especially on the chest. Keloids do not spontaneously resolve, but usually persist for long periods of time, growing relentlessly and can become quite large. In certain situations, keloids may become so large as to be disfiguring. The successful surgical or medical management of keloids is much more difficult than hypertrophic scars, and both may recur after treatment.

Although they may be hard to distinguish from keloids, hypertrophic scars tend to approximate the size of the original wound. Hypertrophic scars tend to be less extensive than normal scars, owing to an increase in the number of peptide cross-linkages present, and they usually spontaneously soften and flatten over a period of time.

3. Pathology of Keloids and Hypertrophic Scars

Both keloids and hypertrophic scars are aberrant forms of wound healing, best understood by comparison with the features seen in ordinary scars.

Although early wound healing results in granulation tissue and epidermal hyperplasia, ordinary scars show a stereotypical pattern consisting of fibroblasts and collagen bundles parallel to the surface of the epidermis and small blood vessels perpendicular to the skin surface. The epidermis overlying a scar maintains a flat ridge pattern for many years. Young scars contain abundant mucopolysaccharides which are minimal in older scars.

Keloid and hypertrophic scars are also raised above the surface of the surrounding skin, and their bulbous contours are often evident in tissue sections.

4. Signs and Symptoms of Keloids and Hypertrophic Scars

Keloids expand in claw-like growths over normal skin. They can cause discomfort, sometimes presenting with a needle-like pain or itch, although the degree of sensation varies from person to person.

If the keloid becomes infected, it may ulcerate. Removing the scar is one treatment option. However, this may result in more severe consequences, since the probability that the resulting surgery scar will also become a keloid is high, usually greater than 50%. Laser treatment has also been used with varying degrees of success.

Keloids form within scar tissue. Collagen, used in wound repair, tends to overgrow in this area, sometimes producing a lump many times larger than that of the original scar. They can also range in color from pink to red. Although they usually occur at the site of an injury, keloids can also arise spontaneously. They can occur at the site of a piercing and even from something as simple as a pimple or scratch. They can occur as a result of severe acne or chicken pox scarring, infection at a wound site, repeated trauma to an area, excessive skin tension during wound closure or due to the presence of a foreign body in a wound. Keloids can sometimes be sensitive to chlorine. Keloid scars can grow, if they appear at a younger age, because the body is still growing.

Hypertrophic scars are thickened, wide, raised scars that develop during the wound healing process, and are the result of an abnormal response to a trauma or injury. Hypertrophic scars can be cosmetically displeasing and can sometimes cause discomfort.

5. Causes of Keloids and Hypertrophic Scars

Most skin injuries can contribute to scarring. This includes burns, acne scars, chicken pox scars, piercings, scratches, surgical cuts, and vaccination sites.

According to the (US) National Center for Biotechnology Information, keloid scarring is common in young people between the ages of 10 and 20. Studies have shown that those with darker complexions are at a higher risk of keloid scarring as a result of skin trauma. They occur in 15%-20% of individuals with African, Asian or Latino ancestry, significantly less often in those of a Caucasian background, and there are no reported cases in patients with albinism. Keloids tend to have a genetic component, which means an individual is more likely to have keloids if one or both parents has them. However, no single gene has yet been identified which is a causing factor in keloid scarring, although several susceptibility loci have been discovered, most notably in Chromosome 15.

Hypertrophic scars are the result of an abnormal response to a trauma or injury. In certain people, myofibroblast cells can produce too much collagen during healing, leading to hypertrophic scarring.

6. Pathogenesis of Keloids and Hypertrophic Scars

Neither keloids nor hypertrophic scars follow the same maturation process as normal scars. Keloids demonstrate accumulation of type I and type VI collagen. Not only is more collagen present in both keloids and hypertrophic scars, this collagen is in a different physical and chemical state. One of the recently described growth factors, TGF-β, has been shown to be capable of activating the production of type I and type VI collagen.

These findings are in marked contrast to the biochemical makeup of collagen found in young scars. The collagen in keloids and hypertrophic scars demonstrates rapid collagen turnover, which is due to the presence of increased embryonic collagen and fibronectin. This may be due to a reduction of collagenase activity. Two other substances, α₂-macrogobulin and α₁-antitrypsin, have been found in high concentrations in keloids. These two substances inhibit collagenase and serve to protect the deposited collagen from enzymatic degradation.

7. Treatments of Keloids and Hypertrophic Scars

The best treatment for keloids and hypertrophic scars is prevention in patients with a known pre-disposition. This includes preventing unnecessary trauma or surgery (including piercings and elective mole removal) whenever possible. Any skin problems in pre-disposed individuals (e.g., acne, infections, etc.) should be treated as early as possible to minimize areas of inflammation.

Treatment of a keloid scar is age dependent. Radiotherapy, anti-metabolites and corticosteroids (frequently also referred to as corticoids) would not be recommended for use in children, in order to avoid harmful side effects (e.g., growth abnormalities).

In adults, corticosteriods can frequently be effective, however, corticosteroids frequently have undesirable side effects, e.g., local over reactions. Corticosteroids, combined with 5-FU (fluorouracil, a pyrimidine analog that is an antineoplastic antimetabolite) and PDL (poly-D-lysine, a lysine homopolymer), in a triple therapy, can sometimes enhance results and diminish side effects.

Further prophylactic and therapeutic strategies include pressure therapy, silicone gel sheeting, intra-lesional triamcinolone acetonide (TAC), cryosurgery, radiation, laser therapy, IFN (interferon), 5-FU and surgical excision, as well as a multitude of extracts and topical agents.

Surgical excision is currently still the most common treatment for a significant amount of keloid lesions. However, when used as the solitary form of treatment, there is a large recurrence rate of between 70% and 100%. It has also been known to cause a larger lesion formation on recurrence. While not always successful alone, surgical excision when combined with other therapies dramatically decreases the recurrence rate. Examples of these therapies include, but are not limited to, radiation therapy, pressure therapy and laser ablation. Pressure therapy following surgical excision has shown promising results, especially in keloids of the ear and earlobe. The mechanism of exactly how pressure therapy works is not presently known, but many patients with keloid scars and lesions have benefited from it.

Another effective treatment for keloids is superficial external beam radiotherapy (SRT), which can achieve cure rates of up to 90%.

Additionally, intralesional injection with a corticosteroid such as Kenalog appears to aid in the reduction of inflammation and pruritus.

Cryotherapy or cryosurgery (i.e., the application of extreme cold) is also used to treat keloids. This treatment method is easy to perform and has shown results with the least chance of recurrence.

Unfortunately, all of the current treatments for keloids and hypertrophic scars suffer from one or more disadvantages.

Accordingly, the primary object of the present invention is to provide a novel pharmaceutical composition for the treatment of keloids, hypertrophic scars and wounds which avoids the disadvantages associated with current methods of treatment, including the undesired side effects of corticoid therapy such as, for example, local over-reactions, and which can be administered easily and successfully and is degraded by the body.

Another object of the present invention is to provide a novel pharmaceutical composition for providing improved skin care.

### SUMMARY OF THE INVENTION

These and other objects of the present invention are achieved by the provision and use of novel pharmaceutical compositions which comprise mixtures of cross-linked glycosaminoglycans (also known as GAGs, or mucopolysaccharides) and taurolidine (and/or taurolidine derivatives, see below). It should be appreciated that, for the purposes of the present invention, the term “glycosaminoglycans” is intended to include both glycosaminoglycans and analogues thereof (note that such analogues are also sometimes referred to as structural analogues, structural analogs, chemical analogues, chemical analogs, and/or simply analogs).

The novel pharmaceutical compositions of the present invention preferably also comprise a carrier within which the mixtures of cross-linked glycosaminoglycans and taurolidine (and/or taurolidine derivatives, see below) reside.

Additionally, other active agents (see below) may also be incorporated into the novel pharmaceutical compositions.

The novel pharmaceutical compositions of the present invention may be in the form of a solution, a gel, a powder, etc.

The novel pharmaceutical compositions of the present invention are preferably injected intralesionally into the target tissue (e.g., the keloids, hypertrophic scars and wounds), although they may also be applied topically.

It is possible, using the novel pharmaceutical compositions of the present invention, to avoid the undesirable side effects of corticoid therapy. More particularly, it has been found that the cross-linked glycosaminoglycans have an inhibitory effect on keloid formation when they are administered non-topically (i.e., intralesionally), and the taurolidine (and/or the taurolidine derivatives, see below) provides an antimicrobial effect to protect the environment of injection, serves as a safety measure with prophylactic benefits, and enhances tissue healing (e.g., by reducing any inflammatory effect of the cross-linked glycosaminoglycans). With the novel pharmaceutical compositions of the present invention, no local over-reactions occur as is the case with the corticoids, and also no negative systemic effects occur. A further advantage is the biological degradability of the cross-linked glycosaminoglycans and taurolidine (and/or taurolidine derivatives, see below) in the body.

Moreover, the pharmaceutical compositions of the present invention also provide successful treatment of deep scar formations in connective tissue such as, for example, Dupuytren's disease of the palmar surfaces or the so-called Induratio penis plastica (IPP) which form without prior injury.

In addition, the novel pharmaceutical compositions of the present invention also provide improved skin care.

In one preferred form of the invention, there is provided a method for treating keloids, hypertrophic scars and/or wounds and/or other skin conditions, the method comprising:

delivering a pharmaceutically effective amount of a pharmaceutical composition to the keloids, hypertrophic scars and/or wounds and/or other skin conditions, wherein the pharmaceutical composition comprises a mixture of cross-linked glycosaminoglycans and taurolidine and/or one or more taurolidine derivatives.

In another preferred form of the invention, there is provided a pharmaceutical composition comprising:

a pharmaceutically effective amount of a mixture of cross-linked glycosaminoglycans and taurolidine and/or one or more taurolidine derivatives.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view showing the mechanism of action for taurolidine.

### DETAILED DESCRIPTION OF THE PREFERRED EMBODIMENTS

1. The Novel Pharmaceutical Compositions in General

The present invention comprises the provision and use of novel pharmaceutical compositions which comprise mixtures of cross-linked glycosaminoglycans (also known as GAGs, or mucopolysaccharides) and taurolidine (and/or taurolidine derivatives, see below). It should be appreciated that, for the purposes of the present invention, the term “glycosaminoglycans” is intended to include both glycosaminoglycans and analogues thereof (note that such analogues are also sometimes referred to as structural analogues, structural analogs, chemical analogues, chemical analogs, and/or simply analogs).

The novel pharmaceutical compositions of the present invention preferably also comprise a carrier within which the mixtures of cross-linked glycosaminoglycans and taurolidine (and/or taurolidine derivatives, see below) reside.

Additionally, other active agents (see below) may also be incorporated into the novel pharmaceutical compositions.

The novel pharmaceutical compositions of the present invention may be in the form of a solution, a gel, a powder, etc.

The novel pharmaceutical compositions of the present invention are preferably injected intralesionally into the target tissue (e.g., the keloids, hypertrophic scars and wounds), although they may also be applied topically.

2. The Cross-Linked Glycosaminoglycans (GAGs)

The cross-linked glycosaminoglycans (GAGs) of the novel pharmaceutical compositions are a good source of collagen, which enhances tissue treatment. Cross-linking the glycosaminoglycans provides higher molecular weights, which prolongs the effectiveness of the pharmaceutical compositions. Note that glycosaminoglycans which are not cross-linked may also be used in the pharmaceutical compositions of the present invention, however, the benefits of prolonged effectiveness are not obtained.

Cross-linked glycosaminoglycans are molecules in which two or several of the same, and/or different, glycosaminoglycans are linked together to form a molecular unit. The cross-linking is preferably carried out by chelate formation, complex formation and/or salt formation, and in particular by a chemical cross-linking.

Natural or synthetic glycosaminoglycans, and also substances that are chemically related thereto, can be used as the cross-linked glycosaminoglycans of the novel pharmaceutical compositions. The glycosaminoglycans can be of an anionic or cationic character.

Where natural glycosaminoglycans are used with the present invention, the glycosaminoglycans are preferably those that occur in human connective tissue, and are preferably hyaluronic acid, heparin, heparin sulfate and chondroitin sulfate.

Where synthetic glycosaminoglycans are used with the present invention, the glycosaminoglycans are preferably sulfated polysaccharides.

Where substances that are chemically related to glycosaminoglycans are used with the present invention, the substances that are chemically related to glycosaminoglycans are preferably those of biological origin, and are preferably chitosamine and chitosan or their derivatives such as, for example, N-carboxybutlychitosan.

It should be appreciated that, for the purposes of the present invention, the formation of chelates, complexes and poorly soluble salts of glycosaminoglycans create a cross-linking of the glycosaminoglycans by a “holding-together” of the glycosaminoglycans (i.e., through ionic forces as is common for such chelates, complexes and salts), whereby to form the cross-linked glycosaminoglycans which are combined with taurolidine (and/or taurolidine derivatives, see below) so as to form the pharmaceutical compositions of the present invention.

3. The Taurolidine (and/or Derivatives of the Taurolidine)

The taurolidine (and/or the taurolidine derivatives) in the novel pharmaceutical compositions of the present invention provides an antimicrobial effect to protect the environment of injection, serve as a safety measure with prophylactic benefits, and enhance tissue healing (e.g., by reducing any inflammatory effect of the cross-linked glycosaminoglycans).

Taurolidine (4,4′-methylene-bis(tetrahydro-1,2,4-thiadiazine)-1,1,1′,1′,-tetraoxide), and/or taurolidine derivatives, is known to have antimicrobial and antilipopolysaccharide properties. Taurolidine (and/or taurolidine derivatives) is also known to exhibit anti-inflammatory properties. The immunomodulatory action of taurolidine (and/or taurolidine derivatives) is reported to be mediated by priming and activation of macrophages and polymorphonuclear leukocytes.

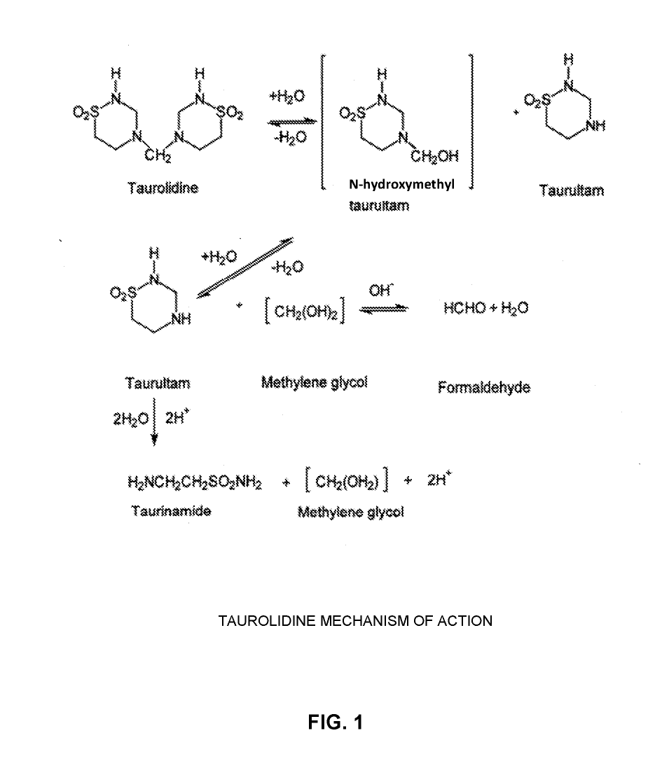

Taurolidine is derived from the amino acid taurine. In aqueous solution, the parent molecule taurolidine forms an equilibrium with N-hydroxymethyl taurultam and taurultam, with taurinamide, methylene glycol and formaldehyde being a downstream derivatives. For the purposes of the present invention, N-hydroxymethyl taurultam, taurultam, taurinamide, methylene glycol and formaldehyde can all be considered taurolidine derivatives. See FIG. 1, which shows taurolidine's mechanism of action.

The active moieties of taurolidine are believed to be the derivative methylol groups which react with the bacterial cell wall, the cell membrane, and the proteins of the cell membrane, as well as with the primary amino groups of endo- and exotoxins. Microbes are killed and the resulting toxins are inactivated; the destruction time in vitro is approximately 30 minutes.

4. Further Details of the Novel Pharmaceutical Compositions of the Present Invention

The novel pharmaceutical compositions of the present invention comprise taurolidine (and/or the taurolidine derivatives) mixed with cross-linked glycosaminoglycans. The novel pharmaceutical compositions of the present invention preferably also comprise a carrier within which the mixtures of taurolidine (and/or taurolidine derivatives) and cross-linked glycosaminoglycans reside. Additionally, other active agents (see below) may also be incorporated into the novel pharmaceutical compositions. The novel pharmaceutical compositions of the present invention may be in the form of a solution, a gel, a powder, etc.

The pharmaceutical compositions are delivered to the keloids, hypertrophic scars and wounds (or other tissue), whereupon the cross-linked glycosaminoglycans provide collagen to the tissue so as to treat the keloids, hypertrophic scars and wounds (e.g., to inhibit the formation of keloids and hypertrophic scars), and the taurolidine (and/or the taurolidine derivatives) protects the environment of injection, serves as a safety measure with prophylactic benefits, and enhances tissue healing (e.g., by reducing any inflammatory effects of the cross-linked glycosaminoglycans). Significantly, by mixing the taurolidine (and/or the taurolidine derivatives) with the cross-linked glycosaminoglycans so that the great majority of the taurolidine (and/or the taurolidine derivatives) is encased by, and hence protected by, the mass of the cross-linked glycosaminoglycans, hydrolysis of the taurolidine (and/or the taurolidine derivatives) is delayed so as to provide a time release effect for the taurolidine (and/or the taurolidine derivatives).

With the novel pharmaceutical compositions of the present invention, the cross-linked glycosaminoglycans preferably constitute approximately 0.1% to approximately 20% by weight of the total pharmaceutical composition, and more preferably constitute approximately 0.5% to approximately 10% by weight of the total pharmaceutical composition, and most preferably constitute approximately 1% to approximately 5% by weight of the total pharmaceutical composition.

With the pharmaceutical compositions of the present invention, the taurolidine preferably constitutes approximately 1% to approximately 10% by weight of the total pharmaceutical composition, and more preferably constitutes approximately 2 to approximately 6% by weight of the total pharmaceutical composition, and most preferably constitutes approximately 3% to approximately 5% by weight of the total pharmaceutical composition.

Additional components may also be included in the novel pharmaceutical compositions. Such additional components of the novel pharmaceutical compositions are discussed in detail below.

5. Cross-Linking the Glycosaminoglycans (GAGs)

The cross-linked glycosaminoglycans (GAGs) used in the pharmaceutical compositions of the present invention are routinely cross-linked with the addition of 1,4-butanediol diglycidyl ether (BDDE). Alternatively, and more preferably, the chemical cross-linking of the glycosaminoglycans is carried out by cross-linking the glycosaminoglycans with bifunctional reactive agents, e.g., glutaraldehyde or carbodiimide. However, it is also possible to produce cross-linking of the glycosaminoglycans via amide bonds by means of bifunctional amino acids such as lysine, protamines or albumins.

If synthetic glycosaminoglycans are used in the pharmaceutical compositions of the present invention, then these may already be primarily cross-linked during production so that further treatment for cross-linking is not necessary. Such synthetic, already primarily cross-linked glycosaminoglycans are commercially available (e.g., “Hylon” and “Hylagel”, a cross-linked hyaluronic acid from the Biomatrix Company, New Jersey, USA) and can be used without further cross-linking when producing the pharmaceutical compositions of the present invention.

It is generally preferable to cross-link identical glycosaminoglycans when forming the pharmaceutical compositions of the present invention, however, it is also possible to use a combination of two or more different (or partially different) glycosaminoglycans when forming the pharmaceutical compositions of the present invention.

In a particularly preferred embodiment of the present invention, glycosaminoglycans with an extremely long chain (e.g., with a molecular weight preferably between 100,000 Daltons and 1,000,000 Daltons) are used; in this case, the degree of cross-linking can then remain low given the starting size of the individual glycosaminoglycan molecules. These products are practically free of protein.

And in a particularly preferred embodiment of the present invention, intermolecularly cross-linked heparin is used as the cross-linked glycosaminoglycan.

The intermolecular cross-linking of heparin can, for example, be carried out in the following manner: commercially available heparin is treated with dilute nitric acid, by which means reactive aldehyde groups are formed on the heparin. Subsequently, a reductive amination (also known as reductive alkylation) is carried out by means of sodium cyanoborohydride (NaBH₃CN). Covalent bonds are formed between the individual heparin chains (end and side chains) via the free amino functional groups of heparin.

In one form of the invention, the cross-linking of heparin is achieved by complex formation, preferably carried out with protamines, gentamycin or vancomycin, or metal ions such as Fe²⁺, Zn²⁺.

In one form of the invention, where the cross-linked glycosaminoglycan comprises heparin, the cross-linked heparin is preferably approximately 0.10% to approximately 3.0% by weight of the pharmaceutical composition.

6. Additional Aspects of the Novel Pharmaceutical Compositions of the Present Invention

The novel pharmaceutical compositions of the present invention can be provided in the form of preparations that can be administered intralesionally and, in particular, in the form of injectable gels preferably having a water content of approximately 80% to approximately 99% by weight, and also as an anhydrous precursor in the form of a powder (e.g., lyophilized powder).

Carrier and pharmaceutical auxiliary substances can be incorporated in the novel pharmaceutical composition. The carrier and pharmaceutical auxiliary substances that can be used with the present invention are those that are:

(i) suitable for the application of the present invention, and

(ii) compatible with the cross-linked glycosaminoglycans and taurolidine.

The preferred carrier substance is water.

The pharmaceutical compositions of the present invention can, for example, contain agents to set the pH level, stabilizers, antioxidants, solubilizers, agents that promote penetration, preservatives and/or gel formers, that is, pharmaceutical auxiliary substances of the sort typically used in pharmaceutical compositions. The pharmaceutical auxiliary substances are preferably used in the typical amounts for such pharmaceutical compositions.

The novel pharmaceutical compositions formed in accordance with the present invention can, in addition to the actual active substances (i.e., the cross-linked glycosaminoglycans and taurolidine (and/or taurolidine derivatives)) also contain further pharmaceutically active substances that are compatible with the cross-linked glycosaminoglycans and taurolidine (and/or taurolidine derivatives), e.g., active substances for the therapy of skin diseases (dermatoses), antibiotics (in particular antibiotics with a charge of opposite polarity such as, for example, gentamycin and vancomycin with a positive charge for cross-linked heparin, or penicillins or cephalosporins with a negative charge for cross-linked chitosamine, etc.), sulfonamides, disinfectants, hormones (e.g., corticoids) and hormone derivatives (e.g., cortisol), local anaesthetics (e.g., of the lidocaine or novocaine type), vasoactive substances for vascular constriction (avoidance of bleeding), adrenalin, enzymes such as hyaluronidase, interleukins, growth factors (e.g., epidermal growth factor (EGF), platelet-derived growth factor (PDGF) and/or insulin-like growth factor (IGF)), skin care agents and/or agents that stimulate blood flow (hyperaemising agents). These substances can be present in the pharmaceutical composition, bound firmly to the glycosaminoglycans (such as antibiotics with a heteropolar charge of opposite polarity as a component of the cross-linked glycosaminoglycans) and then released during the degradation of the cross-linked glycosaminoglycans. Or these substances can be present in the pharmaceutical composition and released by a controlled release type of system, e.g., these substances may be weakly bound to polyethylene glycol (PEG) and then released at the therapy site.

7. Production of the Novel Pharmaceutical Compositions

The novel pharmaceutical compositions of the present invention can be produced in a conventional, generally known manner for the production of pharmaceutical compositions. In this process, the order of mixing of the individual components is generally not critical.

The present invention also concerns a process for the production of the novel pharmaceutical compositions which is characterized in that the cross-linked glycosaminoglycans, the taurolidine (and/or the taurolidine derivatives) and the other components (e.g., pharmaceutical auxiliary substances) may be dissolved in a carrier so as to facilitate delivery to the therapy site. Water is preferably used as the carrier, and it is expedient to carry out the mixing process (i.e., mixing the glycosaminoglycans, taurolidine (and/or taurolidine derivatives) and water) while heating, and subsequently cooling, the mixture. For protection against oxidation, it may be expedient to work in an inert gas environment, in particular in a nitrogen environment.

8. Administration of the Novel Pharmaceutical Compositions

The type, dose and frequency with which the novel pharmaceutical compositions of the present invention is administered depends on the severity of the disease and the general state of the patient, and also on the state and the sensitivity of the scar tissue. If the novel pharmaceutical compositions of the present invention are administered in the form of preparations that can be applied topically, then the administration is, as a rule, in accordance with the usual conditions for topically applying such compositions.

The type of treatment, and the frequency of administration, also depends on the individual response of the person to be treated. An application of gels is preferably carried out at intervals of approximately 1 to approximately 2 months.

9. The Novel Pharmaceutical Compositions in the Form of an Injection

In one preferred form of the present invention, the novel pharmaceutical compositions are in the form of injections, and the pharmaceutical compositions can contain local anesthetics to eliminate pain when the injection needle is inserted. The pharmaceutical compositions preferably also contain an anionically- or cationically-charged molecule such as gentamycin as an antibiotic which is bound, as a counter-ion, to the cross-linked glycosaminoglycans and thus remains immobilized in loco, which prolongs the anesthetic action accordingly.

If the pharmaceutical compositions of the present invention are in the form of injectable gels that are applied intralesionally (which is generally intended to be the case), then this is preferably carried out by injection with the aid of fine needles and pressure-resistant syringes. Pharmaceutical compositions in the form of injectable solutions can also be delivered using fine needles and syringes. The injectable gels of the present invention can also be “shot” percutaneously into the tissue with the aid of compressed air devices; such compressed air devices such as these are well known in medical applications. Pharmaceutical compositions in the form of solutions and fine powders can also be delivered using such compressed air devices.

In a particularly preferred form of the invention, the pharmaceutical compositions are in the form of injectable gels containing cross-linked heparin (as the cross-linked glycosaminoglycan) and taurolidine (and/or taurolidine derivatives). Note that the pharmaceutical compositions do not include non-cross-linked heparin because heparin inhibits blood coagulation and is, additionally, rapidly transported out of the lesion via the vascular system of the patient and then has a detrimental systemic effect similar to that of corticoids. Significantly, heparin loses its inhibitory properties on blood coagulation when heparin is in its cross-linked form and cannot be transported away from the site of application via the blood and lymph stream. Thus, using cross-linked heparin in the pharmaceutical compositions causes the glycosaminoglycans of the pharmaceutical compositions (i.e., the cross-linked heparin) to remain active at the site of application in the tissue for weeks and months after injection. Subsequent degradation of the glycosaminoglycans is primarily via phagocytosis by special cell populations.

Note also that when cross-linked glycosaminoglycans are used in the pharmaceutical compositions of the present invention, there is no occurrence of local over-reactions and no negative systemic effects such as is the case with corticoids.

A further advantage of using cross-linked glycosaminoglycans in the pharmaceutical compositions of the present invention is the biological degradability of the pharmaceutical compositions in the body. Thus, for example, the intralesional application of the pharmaceutical compositions by injection can be repeated at intervals of 4 to 8 weeks.

An additional advantage of providing the novel pharmaceutical compositions of the present invention in the form of injectable gels and their intralesional application is that no additional hygienic measures whatsoever are necessary after the injection sites have healed. All regions of the body can be treated in the same manner and the mobility of the patient is not restricted by bandages.

Treatment with the novel pharmaceutical compositions of the present invention can also prevent an occurrence or re-occurrence of keloids which demonstrates its preventive effect. Thus it will be seen that the novel pharmaceutical compositions of the present invention are not only useful to treat skin conditions, they are also useful to inhibit or prevent the occurrence of skin conditions.

The present invention, therefore, relates to the provision and use of novel mixtures of cross-linked glycosaminoglycans and taurolidine (and/or taurolidine derivatives) for the treatment of keloids, hypertrophic scars and wounds, and for the treatment of other skin conditions.

10. Novel Pharmaceutical Compositions in the Form of an Anhydrous Precursor

In one form of the invention, the novel pharmaceutical compositions of the present invention are administered in the form of anhydrous precursors of the cross-linked glycosaminoglycans (e.g., as a lyophilisate) and the taurolidine (and/or the taurolidine derivatives), e.g., in the form of a wound powder for disposition into fresh wounds. In this case, the powder form of the novel pharmaceutical compositions is sprinkled into the open wound or wound cavity before wound closure. The wound is then closed by sutures, staples, clamps, etc. In the wound, the anhydrous powder of the novel pharmaceutical compositions takes up water from the tissue so as to create the hydrated form of the novel pharmaceutical compositions (e.g., so that it takes the form of a gel or a solution at the wound site).

The powder or gel form of the novel pharmaceutical compositions can also be introduced into large body cavities to prevent undesired adhesions, e.g., the novel pharmaceutical compositions can be introduced into the abdominal cavity or thoracic cavity of a patient during a surgical operation on the intestine or the lung, into the pericardium of a patient during a surgical procedure, or into the body of a patient after operative procedures (e.g., via indwelling drainages). In the case of inflammatory effusions into large body cavities, the novel pharmaceutical compositions of the present invention can also be used to treat the inflammatory effusions by introducing the novel pharmaceutical compositions into the body cavities via an indwelling cannula after puncturing into the body cavity and emptying the effusion.

The novel pharmaceutical compositions of the present invention can also be introduced into cavities and ducts of the body that are accessible from the outside, e.g., into the main nasal cavities and paranasal sinuses, or into the meati of the nose, or into the tear ducts, so as to prevent scarred adhesions.

11. Delivery of the Novel Pharmaceutical Compositions Via a Tampon, Catheter, Bandage, Etc.

The novel pharmaceutical compositions of the present invention may also be disposed on a suitable physical article such as a tampon, catheter, bandage, etc. In this way, when the physical article is disposed against tissue, the novel pharmaceutical compositions will be delivered, via physical contact, to the tissue.

12. Use of the Novel Pharmaceutical Compositions in Cosmetic and Skin Care Preparations

It is known from U.S. Pat. No. 4,605,691 that cross-linked gels of hyaluronic acid can be used alone, or together with other hydrophilic polymers, in cosmetic formulations. R. Muzzarelli et al., Carbohydrate Polymers 11 (1989) 307-320, also describes the use of N-carboxybutylchitosan for cosmetic preparations.

It has now, surprisingly, been found that the pharmaceutical compositions of the present invention (comprising a mixture of cross-linked glycosaminoglycans and taurolidine, and/or taurolidine derivatives) are highly suitable as a carrier of, or a component of, cosmetics and skin care products.

The present invention, therefore, also concerns the use of the novel pharmaceutical compositions (comprising a mixture of cross-linked glycosaminoglycans and taurolidine, and/or taurolidine derivatives) for cosmetics or as skin care products.

The cosmetic preparations and skin care products can be present in the usual forms for such cosmetic preparations and skin care products (e.g., as creams, ointments, lotions and, in particular, gels) that can be applied topically. They can also contain other auxiliary and/or carrier substances that are conventionally used for cosmetic preparations and skin care products which are compatible with the cross-linked glycosaminoglycans and taurolidine (and/or taurolidine derivatives) of the present invention. In addition, cosmetic preparations and skin care products incorporating the novel pharmaceutical compositions of the present invention can also contain auxiliary substances and/or further active substances, provided that they are compatible with the cross-linked glycosaminoglycans and taurolidine (and/or taurolidine derivatives).

### Modifications

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A method for treating keloids, hypertrophic scars and/or wounds and/or other skin conditions, the method comprising:
delivering a pharmaceutically effective amount of a pharmaceutical composition to the keloids, hypertrophic scars and/or wounds and/or other skin conditions, wherein the pharmaceutical composition comprises a mixture of cross-linked glycosaminoglycans and taurolidine and/or one or more taurolidine derivatives.

2. A method according to claim 1 wherein the glycosaminoglycans used to form the cross-linked glycosaminoglycans comprise at least one from the group consisting of hyaluronic acid, heparin, heparin sulfate, chondroitin sulfate and sulfated polysaccharides.

3. A method according to claim 1 wherein the cross-linked glycosaminoglycans are formed from a single type of glycosaminoglycans.

4. A method according to claim 1 wherein the cross-linked glycosaminoglycans are formed from at least two different types of glycosaminoglycans.

5. A method according to claim 1 wherein the cross-linked glycosaminoglycans comprise cross-linked hyaluronic acid.

6. A method according to claim 5 wherein the cross-linked hyaluronic acid is approximately 0.1% to approximately 20% by weight of the pharmaceutical composition.

7. A method according to claim 1 wherein the cross-linked glycosaminoglycans comprise cross-linked heparin.

8. A method according to claim 7 wherein the cross-linked heparin is approximately 0.10% to approximately 3.0% by weight of the pharmaceutical composition.

9. A method according to claim 1 wherein the cross-linked glycosaminoglycans are cross-linked by chemical cross-linking, chelate formation, complex formation or salt formation.

10. A method according to claim 1 wherein the taurolidine and/or the one or more taurolidine derivatives is approximately 1% to approximately 10% by weight of the pharmaceutical composition.

11. A method according to claim 1 wherein the pharmaceutical composition further comprises a carrier within which the mixture of the cross-linked glycosaminoglycans and taurolidine and/or one or more taurolidine derivatives resides.

12. A method according to claim 11 wherein the carrier comprises water.

13. A method according to claim 1 wherein the pharmaceutical composition is in the form of a solution.

14. A method according to claim 1 wherein the pharmaceutical composition is in the form of a gel.

15. A method according to claim 1 wherein the pharmaceutical composition is in the form of a powder.

16. A method according to claim 1 wherein the pharmaceutical composition is delivered by injection.

17. A method according to claim 1 wherein the pharmaceutical composition is delivered by topical application.

18. A pharmaceutical composition comprising:
a pharmaceutically effective amount of a mixture of cross-linked glycosaminoglycans and taurolidine and/or one or more taurolidine derivatives.

19. A pharmaceutical composition according to claim 18 wherein the glycosaminoglycans used to form the cross-linked glycosaminoglycans comprise at least one from the group consisting of hyaluronic acid, heparin, heparin sulfate, chondroitin sulfate and sulfated polysaccharides.

20. A pharmaceutical composition according to claim 18 wherein the cross-linked glycosaminoglycans are formed from a single type of glycosaminoglycans.

21. A pharmaceutical composition according to claim 18 wherein the cross-linked glycosaminoglycans are formed from at least two different types of glycosaminoglycans.

22. A pharmaceutical composition according to claim 18 wherein the cross-linked glycosaminoglycans comprise cross-linked hyaluronic acid.

23. A pharmaceutical composition according to claim 22 wherein the cross-linked hyaluronic acid is approximately 0.1% to approximately 20% by weight of the pharmaceutical composition.

24. A pharmaceutical composition according to claim 18 wherein the cross-linked glycosaminoglycans comprise cross-linked heparin.

25. A pharmaceutical composition according to claim 24 wherein the cross-linked heparin is approximately 0.10% to approximately 3.0% by weight of the pharmaceutical composition.

26. A pharmaceutical composition according to claim 18 wherein the cross-linked glycosaminoglycans are cross-linked by chemical cross-linking, chelate formation, complex formation or salt formation.

27. A pharmaceutical composition according to claim 18 wherein the taurolidine and/or the one or more taurolidine derivatives is approximately 1% to approximately 10% by weight of the pharmaceutical composition.

28. A pharmaceutical composition according to claim 18 wherein the pharmaceutical composition further comprises a carrier within which the mixture of the cross-linked glycosaminoglycans and taurolidine and/or one or more taurolidine derivatives resides.

29. A pharmaceutical composition according to claim 28 wherein the carrier comprises water.

30. A pharmaceutical composition according to claim 18 wherein the pharmaceutical composition is in the form of a solution.

31. A pharmaceutical composition according to claim 18 wherein the pharmaceutical composition is in the form of a gel.

32. A pharmaceutical composition according to claim 18 wherein the pharmaceutical composition is in the form of a powder.
